(19) United States
(12) Patent Application Publication
(10) Pub. No.: US 2024/0025847 A1
(43) Pub. Date: Jan. 25, 2024

(54) METHOD FOR PREPARING MERCAPTANS BY SULFHYDROLYSIS OF SULFIDES

(71) Applicant: Arkema France, Colombes (FR)

(72) Inventors: Georges Fremy, Lacq (FR); Hélori Salembier, Lacq (FR); Guillaume Laudumiey, Lacq (FR)

(73) Assignee: Arkema France, Colombes (FR)

(21) Appl. No.: 17/766,456

(22) PCT Filed: Sep. 28, 2020
(86) PCT No.: PCT/FR2020/051682

§ 371 Date: Apr. 4, 2022

(30) Foreign Application Priority Data
Oct. 4, 2019 (FR) 1911005

(51) Int. Cl. C07C 319/06; C07C 319/28; B01J 21/06

(52) U.S. Cl. CPC C07C 319/06; C07C 319/28; B01J 21/066; B01J 21/063

(57) ABSTRACT

The present invention concerns a method for sulfhydrolysis from dialkyl sulfides and hydrogen sulfide, in the presence of a specific catalyst based on titanium dioxide and/or zirconium dioxide, as well as the corresponding use of such a catalyst. The present invention also concerns a method for preparing mercaptans, in particular methyl mercaptan, from at least one alcohol and hydrogen sulfide, comprising said sulfhydrolysis method.

### CROSS-REFERENCE TO RELATED APPLICATIONS

This application is the U.S. national phase of International Application No. PCT/FR2020/051682, filed 28 Sep. 2020, which claims priority to French Application No. FR 1911005, filed 4 Oct. 2019, the disclosure of each of these applications being incorporated herein by reference in its entirety for all purposes.

### FIELD OF THE INVENTION

The present invention relates to a process for preparing mercaptans, in particular methyl mercaptan, from dialkyl sulfides and hydrogen sulfide (also known as the sulfhydrolysis process or reaction), in the presence of a specific catalyst based on titanium dioxide and/or zirconium dioxide, and also to the corresponding use of such a catalyst.

The present invention also relates to a process for preparing mercaptans and dialkyl sulfides, from at least one alcohol and hydrogen sulfide, involving the sulfhydrolysis process as defined above.

### BACKGROUND OF THE INVENTION

Mercaptans are of great interest industrially and are currently in widespread use in the chemical industries, notably as starting materials in the synthesis of more complex organic molecules. For example, methyl mercaptan (CH₃SH) is used as a starting material in the synthesis of methionine, an essential amino acid for animal nutrition. Methyl mercaptan is also used in the synthesis of dialkyl disulfides, in particular in the synthesis of dimethyl disulfide (DMDS), a sulfiding additive for hydrotreating catalysts for petroleum fractions, among other applications.

Mercaptans, and in particular methyl mercaptan, are generally synthesized industrially by a known process starting from an alcohol and hydrogen sulfide at elevated temperature in the presence of a catalyst according to equation (1) below:

Main reaction

ROH+H₂S—>RSH+H₂O (1)

However, this reaction gives rise to the formation of byproducts, such as sulfides (which are symmetrical in the case below) according to equation (2) below:

ROH+RSH—>RSR+H₂O (2)

Furthermore, when the main reaction is performed in the presence of several alcohols, dissymmetrical sulfides may also be obtained according to equations (3) and (4) below (example given with two alcohols):

ROH+R′OH+2H₂S—>RSH+R′SH+2H₂O (3)

ROH+R′SH—>RSR′+H₂O (4)

The symmetrical or dissymmetrical sulfide byproducts are obtained in large amount industrially and are primarily sent for destruction. This represents a loss of efficiency in the mercaptan production process and an added cost associated with destroying them.

Thus, sulfides are occasionally upgraded to obtain the corresponding mercaptans, by means of the following reaction (5) (also known as sulfhydrolysis):

Sulfhydrolysis reaction

RSR′+H₂S—>RSH+R′SH (5)

In the case of methyl mercaptan, the sulfhydrolysis reaction is written according to equation (6) below:

CH₃SCH₃+H₂S—>2CH₃SH (6)

The sulfhydrolysis reaction is generally catalyzed with catalysts of alumina type (Al₂O₃) or of NiMo (Nickel/Molybdenum) or CoMo (Cobalt/Molybdenum) type on an alumina support, as described in patent applications WO 2017/210070 and WO 2018/035316.

However, such a process needs to be improved so as to be more economical and more suited to the industrial scale.

There is thus a need for an improved process for the sulfhydrolysis of sulfides to mercaptans, in particular of dimethyl sulfide to methyl mercaptan.

There is also a need for an improved process for upgrading sulfides, in particular dimethyl sulfide, which are generated as byproducts during the production of mercaptans produced from alcohol(s) and hydrogen sulfide.

### SUMMARY OF THE INVENTION

One objective of the present invention is to propose a catalyst for the sulfhydrolysis of sulfides to mercaptans which is easy to perform, economical and gives a satisfactory conversion.

Another objective of the present invention is to propose a process for the sulfhydrolysis of sulfides to mercaptans which can be readily integrated into a unit for the industrial production of mercaptans, notably produced from alcohol(s) and H₂S.

One objective of the present invention is to provide a process for preparing mercaptans in which the sulfides generated as byproducts (for example during the reaction between an alcohol and H₂S) are recycled or economically upgraded, easy to use and industrially viable.

### BRIEF DESCRIPTION OF THE DRAWINGS

FIG. 1 illustrates an embodiment of a methyl mercaptan production unit incorporating the sulfhydrolysis process according to the invention.

FIG. 2 illustrates another embodiment of a methyl mercaptan production unit incorporating the sulfhydrolysis process according to the invention.

FIG. 3 illustrates the percentage selectivity toward sulfides for three different reaction temperatures as a function of the mass percentage of mercaptans in the feedstock entering the main mercaptan synthesis reactor.

### DETAILED DESCRIPTION OF THE INVENTION

The present inventors have discovered, surprisingly, that the use of a specific catalyst, based on titanium dioxide (of formula TiO₂) and/or zirconia (also known as zirconium dioxide, of formula ZrO₂) during sulfhydrolysis makes it possible to obtain a good conversion of sulfides, notably a conversion of at least 36%, preferably of at least 50%, or even of at least 70%. Furthermore, methane as a sulfhydrolysis byproduct is produced with very low selectivity, for example less than 2%.

The catalysts according to the invention are known as inert catalytic supports (i.e. supports with no catalytic activity). They are thus simple, economical and sparingly harmful compositions, making it possible to obtain a more efficient and more environmentally friendly sulfhydrolysis process.

In particular, the sulfhydrolysis process according to the invention may be integrated into a plant for the industrial production of mercaptans, notably produced from at least one alcohol and H₂S. The sulfhydrolysis process according to the invention then makes it possible to increase the mercaptan productivity in a simple and economical manner by upgrading the sulfides generated as byproducts during the main reaction and transforming them also into mercaptans.

Furthermore, surprisingly, the present inventors have also discovered that the mercaptans derived from the sulfhydrolysis and the unreacted H₂S could be reintroduced directly (notably without a separation and/or purification step) into the main reactor, and without this having any consequence on the main reaction between the alcohol(s) and the H₂S.

The mercaptans produced by the two reactions (main reaction and sulfhydrolysis reaction) can then be separated and/or purified and/or recovered in a single place, for example at the outlet of the main reactor.

This integration of the sulfhydrolysis process into the main mercaptan production chain can be reinforced by the presence of a single H₂S feed for both the main reaction and the sulfhydrolysis reaction (for example at the inlet of the sulfhydrolysis reactor).

Thus, according to the invention, a simple and efficient process for the upgrading of sulfides which is totally integrated into an industrial mercaptan production chain may be obtained. This device is notably easy to implement: it can be readily connected to the main unit and requires only minimal modifications thereof.

Thus, the present invention relates to a sulfhydrolysis process, in which a sulfide, preferably a dialkyl sulfide, is reacted with hydrogen sulfide (H₂S) in the presence of ZrO₂ and/or TiO₂ as catalyst(s), to obtain at least one mercaptan, preferably one mercaptan.

The present invention also relates to a mercaptan preparation process comprising the steps of:
preparing mercaptan(s) and dialkyl sulfide(s) from at least one alcohol and H₂S, and
reacting said dialkyl sulfide(s) produced with H₂S according to the sulfhydrolysis process according to the invention, to obtain said mercaptan(s).

### Definitions

The term “catalyst” notably means a substance or a composition of chemical substances which accelerate a chemical reaction and which are unchanged at the end of this reaction.

According to the present invention, the catalyst used in the sulfhydrolysis reaction comprises titanium dioxide (TiO₂) and/or zirconia (ZrO₂), preferably titanium dioxide. Such catalysts may also be referred to as catalysts based on titanium dioxide and/or zirconia.

Titanium dioxide and/or zirconia are used as catalyst(s) in the sulfhydrolysis reaction. It is understood that TiO₂ and/or ZrO₂ are the active components of the catalyst (i.e. the compounds with catalytic activity). In particular, the catalysts according to the invention do not comprise any other compounds which have catalytic activity on the sulfhydrolysis reaction.

Preferably, the catalysts according to the invention consists essentially of, or even consist of, titanium dioxide and/or zirconia, and optionally stabilizers and/or binders. The stabilizers and binders are those conventionally used in the field of catalysts.

The term “promoter” (also known as a “dopant”) is a chemical substance or a composition of chemical substances which can modify and notably improve the catalytic activity of a catalyst. For example, the term “promoter” means a chemical substance or a composition of chemical substances for improving the conversion and/or selectivity of the catalyzed reaction relative to the catalyst alone. Preferably, the catalysts according to the invention do not comprise any promoter.

The usual definitions of conversion, of selectivity and of yield are as follows:
Conversion=(number of moles of reagent in the initial state−number of moles of reagent remaining after the reaction)/(number of moles of reagent in the initial state)
Selectivity=Number of moles of reagent converted into the desired product/(number of moles of reagent in the initial state−number of moles of reagent remaining after the reaction)
Yield=conversion×selectivity

In particular, the sulfhydrolysis catalysts according to the invention make it possible to obtain a sulfide conversion of between 30% and 90%, preferably between 50% and 80% and even more preferentially between 50% and 75%.

The selectivity of the sulfhydrolysis reaction for mercaptans is notably greater than or equal to 98%.

### Sulfides and Mercaptans

The term “sulfide” means any organic compound comprising a —C—S—C— function.

Preferably, the term “sulfide” means a dialkyl sulfide.

The term “dialkyl sulfide” notably means a compound of general formula (I) below:

R—S—R′ (I)

in which R and R′, which may be identical or different, are, independently of each other, a saturated, linear, branched or cyclic, optionally substituted hydrocarbon-based radical.

Preferably, R and R′, which may be identical or different, are, independently of each other, a linear or branched alkyl radical; more preferentially, a linear or branched, preferably linear, alkyl radical containing between 1 and 18 carbon atoms, preferably between 1 and 12 carbon atoms.

R and R′, which may be identical or different, may be chosen, independently of each other, from the group consisting of methyl, ethyl, propyl, butyl, pentyl, hexyl, heptyl, octyl, nonyl, decyl, undecyl and dodecyl, and also isomers thereof. Preferably, R and R′, which may be identical or different, may be chosen, independently of each other, from the group consisting of methyl, ethyl, octyl and dodecyl.

Preferably, R and R′ are identical (which corresponds to a symmetrical dialkyl sulfide).

Symmetrical dialkyl sulfides in particular have the general formula (II) below:

R—S—R (II)

in which R is as defined above.

In particular, the dialkyl sulfides according to the invention are chosen from the group consisting of dimethyl sulfide, diethyl sulfide, dioctyl sulfide, didodecyl sulfide and methyl ethyl sulfide. The dialkyl sulfides according to the invention may be chosen from the group consisting of dimethyl sulfide, diethyl sulfide, dioctyl sulfide and didodecyl sulfide. Most particularly preferably, the dialkyl sulfide is dimethyl sulfide.

The mercaptans according to the invention are those corresponding to the sulfhydrolysis of the sulfides as defined above. Preferably, the term “mercaptans” means alkyl mercaptans.

In particular, the term “alkyl mercaptan” means a compound of general formula (III) and/or (IV) below:

R—SH (III) and/or

R′SH (IV),

in which R and R′ are as defined above.

Particularly preferably, the mercaptan obtained according to the invention is methyl mercaptan.

### Process for the Sulfhydrolysis of Sulfides to Mercaptans

The present invention thus relates to a process for preparing at least one mercaptan by sulfhydrolysis. In particular, the present invention relates to a process for preparing at least one mercaptan, in which a dialkyl sulfide is reacted with hydrogen sulfide (H₂S) in the presence of ZrO₂ and/or TiO₂ as catalyst(s).

Preferably, said catalyst does not comprise any alkali metal oxide notably such as lithium, sodium, potassium, rubidium and cesium oxides.

Preferably, said catalyst does not comprise any promoter.

In particular, said catalyst does not comprise any alumina (Al₂O₃).

In particular, said catalyst does not comprise any phosphorus.

Preferably, said catalyst is TiO₂. Particularly preferably, the catalyst comprises only TiO₂ as active component, and in particular in its anatase crystalline form.

When the catalyst comprises TiO₂ and/or ZrO₂, it may then comprise between 30% and 50%, preferably between 35% and 45%, for example about 40% by weight of TiO₂ relative to the total weight of the catalyst and/or between 50% and 70%, preferably between 55% and 65%, for example about 60% by weight of ZrO₂ relative to the total weight of the catalyst.

The catalysts according to the invention may have a specific area of greater than 40 m²·g⁻¹. Preferably, the specific area is at least 50 m²·g⁻¹ for a catalyst based on ZrO₂. Preferably, the specific area is at least 80 m²·g⁻¹ for a catalyst based on TiO₂.

The form of the catalysts may be of any type, for example spherical, cylindrical, ring-shaped or star-shaped, in the form of granulates or in any other three-dimensional form, or alternatively in the form of a powder which may be pressed, extruded or granulated.

The sulfhydrolysis reagents may be in gaseous, liquid or solid form, preferably gaseous or liquid form.

The sulfhydrolysis reaction temperature may be comprised between 100° C. and 500° C., preferably between 200° C. and 400° C., more preferably between 200° C. and 380° C. and more preferentially between 250° C. and 380° C.

The sulfhydrolysis reaction may be performed at a pressure of between 50 mbar and 100 bar, preferably between atmospheric pressure (about 1 bar) and 50 bar, and advantageously between 5 and 20 bar.

The H₂S/sulfide mole ratio may be comprised between 0.1/1 and 50/1, preferably between 2/1 and 20/1. Preferably said ratio is between 2/1 and 15/1, more preferably between 2/1 and 10/1, for example 4/1.

Advantageously, the reagents (sulfide and H₂S) may respect a particular contact time with the catalyst in the reactor where the sulfhydrolysis takes place. This parameter is expressed with the hourly space velocity equation:

(HSV)=(total flow rate of sulfide+H₂S entering)/(volume of catalyst in the reactor).

The HSV may be comprised between 100 and 1200 h⁻¹.

The sulfhydrolysis reaction may take place in any type of reactor, for example fixed-bed tube reactors, multitubular reactors, with microchannels, with a catalytic wall or with a fluidized bed, preferably a fixed-bed tube reactor.

In particular, the sulfhydrolysis process according to the invention is performed in a reactor comprising only one catalytic zone (said zone is notably continuous).

The amount of each reagent supplied to the reactor may vary as a function of the reaction conditions (for example the temperature, the hourly space velocity, etc.) and is determined according to the general knowledge. The hydrogen sulfide may be present in excess.

### Process for Producing Mercaptans

The present invention relates to a process for preparing mercaptan(s) and dialkyl sulfide(s) from at least one alcohol and H₂S, in which said dialkyl sulfide(s) produced then react with H₂S according to the sulfhydrolysis process as defined above, to obtain said mercaptan(s).

Thus, the present invention relates to a process for preparing mercaptan(s), comprising the steps of:
preparing mercaptan(s) and dialkyl sulfide(s) from at least one alcohol and H₂S, and
reacting said dialkyl sulfide(s) produced with H₂S according to the sulfhydrolysis process as defined above, to obtain said mercaptan(s).

The reaction between an alcohol and H₂S to form a mercaptan (and a sulfide as byproduct) is a known reaction, described, for example, in patents U.S. Pat. Nos. 2,820,062 A, 7,645,906 B2 and 2,820,831 A. For example, the reaction may be performed at a temperature of between 200° C. and 450° C. and/or at a pressure ranging from a reduced pressure to 100 bar. Generally, a catalyst is present, such as alumina promoted by alkali metals and/or alkaline-earth metals. The H₂S may be present in excess.

Among the reagents, at least one alcohol, preferably one or two alcohols, may be used. Preferentially, only one alcohol is used. The alcohol(s) may be chosen from (C₁-C₁₈) or even (C₁-C₁₂) alcohols, and mixtures thereof. In particular, the alcohols may be chosen from the group consisting of methanol, ethanol, octanol, dodecanol and mixtures thereof. Preferably, the alcohol used is methanol.

Hereinbelow, it is understood that when two streams are introduced into a reactor, they may each be introduced separately into the reactor or may be combined before being introduced into the reactor.

In particular, the process according to the invention comprises the following steps:

A) introducing a stream comprising H₂S and a stream comprising at least one alcohol into a first reactor;

B) reacting the two streams to obtain an outlet stream comprising at least one mercaptan, at least one dialkyl sulfide and optionally H₂S;

C) separating the outlet stream obtained from step B) into:
a stream comprising the mercaptan(s),
a stream comprising the dialkyl sulfide(s), and
optionally a stream comprising H₂S;

D) introducing said stream comprising the dialkyl sulfide(s) into a second reactor with a stream of H₂S;

E) reacting the two streams according to the sulfhydrolysis process as defined above to obtain an outlet stream comprising said mercaptan(s) and optionally H₂S;

F) optionally recycling the stream of H₂S obtained from step C) into step A).

The mercaptan(s) may be recovered on conclusion of step C) and/or after separation of the outlet stream from step E), preferably on conclusion of step C).

The outlet stream obtained from step B) may comprise at least one mercaptan, at least one dialkyl sulfide, optionally water, unconverted alcohol(s) and H₂S. The outlet stream obtained from step E) may comprise said mercaptan(s), H₂S and optionally methane and the unconverted dialkyl sulfide(s).

The outlet stream from the second reactor of step E) may be recycled, preferably entirely, into the first reactor of step A). Thus, the outlet stream from the sulfhydrolysis process comprising the mercaptan(s) and optionally the H₂S may be introduced directly into the main reactor (or first reactor), notably without a prior separation and/or purification step. In particular, the outlet stream from the second reactor of step E) may correspond entirely or partially, preferably entirely, to the stream comprising H₂S from step A), optionally with recycled H₂S obtained from step C). In this case, the outlet stream obtained from step E) comprises H₂S.

Surprisingly, the introduction of mercaptans into the main reactor has no influence on the main reaction between at least one alcohol and H₂S. Furthermore, the H₂S which may be obtained from step E) is thus totally recycled into step A). Such recycling notably has the advantage of having only one H₂S inlet for the entire mercaptan production process, for example at the inlet of the sulfhydrolysis reactor.

Thus, the sulfhydrolysis process according to the invention integrated into an industrial mercaptan production facility makes it possible to totally reprocess the sulfide byproducts as products of interest and advantageously to recycle the H₂S. The mercaptans produced will be the result of the main reaction and of the sulfhydrolysis reaction, which increases the productivity.

The separation step C) may be performed by distillation, for example under reduced pressure, according to conventional methods. For example, the distillation may take place at a pressure of between 0.1 bar and 10 bar, notably between 1 and 10 bar. Step C) may notably make it possible to separate the outlet stream obtained from step B) into:

a stream comprising the mercaptan(s),

a stream comprising the dialkyl sulfide(s),

optionally a stream comprising H₂S,

optionally a stream comprising methane,

optionally water,

optionally unconverted alcohol.

According to a particular embodiment, said preparation process comprises the following steps:

a) introducing a stream comprising a mercaptan and H₂S and a stream comprising at least one alcohol, preferably one alcohol, into a first reactor;
b) reacting the two streams to obtain an outlet stream comprising the mercaptan, a dialkyl sulfide and optionally H₂S;
c) separating the outlet stream obtained from step b) into:
a stream comprising the mercaptan;
a stream comprising the dialkyl sulfide; and
optionally a stream comprising H₂S;

d) introducing the stream comprising the dialkyl sulfide into a second reactor with a stream of H₂S;

e) reacting the two streams according to the sulfhydrolysis process as defined above to obtain an outlet stream comprising the mercaptan and H₂S;

f) introducing the outlet stream obtained from step e), preferably directly, into the reactor of step a);

g) optionally recovering the stream comprising the mercaptan obtained from step c); and

h) optionally recycling the stream of H₂S obtained from step c) into step a).

The outlet stream from step f) may correspond entirely or partially, preferably entirely, to the inlet stream of mercaptan and H₂S of step a).

According to another particular embodiment, said preparation process comprises the following steps:

a′) introducing a stream comprising H₂S and a stream comprising at least one alcohol, preferably one alcohol, into a first reactor;

b′) reacting the two streams to obtain an outlet stream comprising a mercaptan, a dialkyl sulfide and optionally H₂S;

c′) separating the outlet stream obtained from step b′) into:
a stream comprising the mercaptan;
a stream comprising the dialkyl sulfide; and
optionally a stream comprising H₂S;

d′) optionally recovering the stream comprising the mercaptan obtained from step c′);

e′) optionally recycling the stream of H₂S obtained from step c′) into step a′);

f′) introducing the stream comprising the dialkyl sulfide into a second reactor with a stream of H₂S;

g′) reacting the two streams according to the sulfhydrolysis process as defined above to obtain an outlet stream comprising the mercaptan and H₂S;

h′) separating the outlet stream obtained from step g′) into:
a stream comprising the mercaptan;
a stream comprising H₂S;

i′) combining the stream comprising the mercaptan obtained from step h′) with the outlet stream from step b′) before and/or during step c′);

j′) optionally recycling the stream of H₂S obtained from step h′) into step f′).

This embodiment notably offers independence of the H₂S feed of the main reactor relative to the sulfhydrolysis reactor.

The present invention also relates to the use of ZrO₂ and/or TiO₂ as catalyst(s), in the reaction reacting a sulfide with hydrogen sulfide to obtain a mercaptan. In particular, said catalyst and said reaction are as defined for the sulfhydrolysis process as described above. The sulfides and mercaptans are also as defined above.

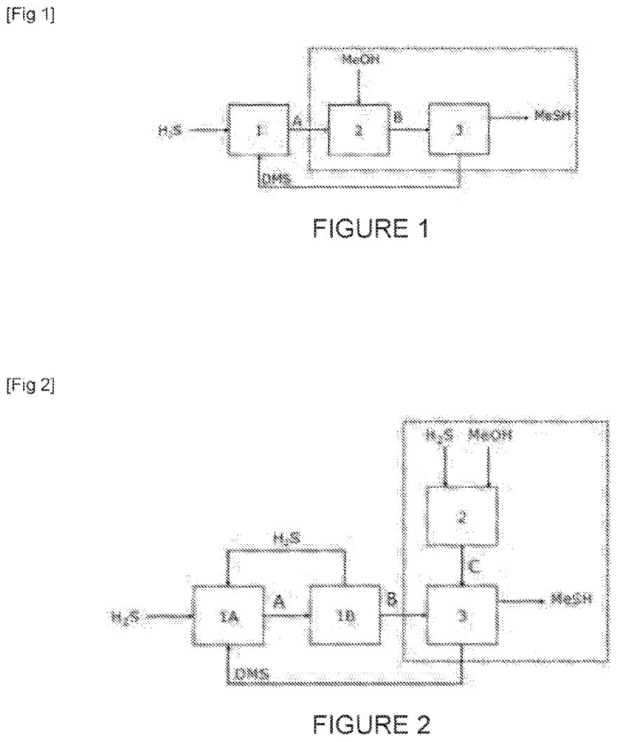

FIG. 1 schematically shows a methyl mercaptan production unit incorporating the sulfhydrolysis process according to the invention. The production unit may be pre-existing and may correspond to the elements surrounded by dashed lines.

The secondary reactor (1) (where the sulfhydrolysis takes place) comprises an H₂S inlet and a dimethyl sulfide (DMS) inlet. At the outlet, stream A comprises H₂S and methyl mercaptan.

Stream A enters directly into the main reactor (2) also comprising a methanol inlet. The outlet stream B from the reactor (2) comprises methyl mercaptan, dimethyl sulfide and H₂S.

Stream B is then separated by distillation (3) into three different streams:

a stream comprising methyl mercaptan (MeSH);

a stream comprising dimethyl sulfide (DMS); and

a stream comprising H₂S, which is subsequently recycled into the reactor (2) (not shown).

The DMS stream is then recycled into the reactor (1).
FIG. 2 schematically shows another embodiment of a methyl mercaptan production unit incorporating the sulfhydrolysis process according to the invention. The production unit may be pre-existing and may correspond to the elements surrounded by dashed lines.

The secondary reactor (1A) (where the sulfhydrolysis takes place) comprises an H₂S inlet and a dimethyl sulfide (DMS) inlet. At the outlet, stream A comprises H₂S and methyl mercaptan.

Stream A undergoes a step of separation by distillation at (1B), which gives rise to a stream of H₂S recycled into the reactor (1A) and a stream B which comprises methyl mercaptan which is combined at (3) with the outlet stream C from the reactor (2), where the main reaction takes place between methanol and H₂S.

A step of separation by distillation (3) gives two different streams:
a stream comprising methyl mercaptan (MeSH);
a stream comprising dimethyl sulfide (DMS).

The DMS stream is then recycled into the reactor (1A).

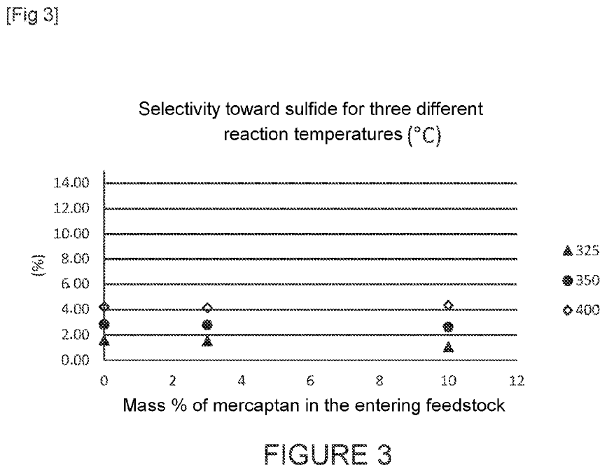

FIG. 3 represents the percentage selectivity toward sulfides for three different reaction temperatures as a function of the mass percentage of mercaptans in the feedstock entering the main mercaptan synthesis reactor.

### EXAMPLES

### Example 1: Process for the Sulfhydrolysis of Dimethyl Sulfide (DMS) to Methyl Mercaptan (MeSH)

Before the test, the catalysts were activated in situ by means of a procedure comprising a first step of drying with nitrogen at 250° C., followed by sulfidation with H₂S at 350° C. for 1 hour.

The performance of the catalysts was evaluated for the reaction for producing MeSH from DMS in a fixed-bed reactor comprising only one catalytic zone with a catalyst volume of 30 mL, a temperature ranging from 300° C. to 350° C., under a pressure of 9 bar absolute, with a feed gas composition DMS/H₂S=¼ (v/v) and an hourly space velocity of 800 h⁻¹. The reagents are preheated to a temperature >100° C. and are flashed during their introduction into the bottom of the reactor.

The products were analyzed online by gas chromatography.

The results for the DMS conversion and the selectivity toward MeSH and CH₄ obtained for three different catalysts are described in Table 1 below:

TABLE 1

| | Al₂O₂ | | | ZrO₂ | | | TiO₂ | | |
|---|---|---|---|---|---|---|---|---|---|
| | Conversion | Selectivities | | Conversion | Selectivities | | Conversion | Selectivities | |
| T | (%) | (%) | | (%) | (%) | | (%) | (%) | |
| (° C.) | (CH₃)₂S | CH₃SH | CH₄ | (CH₃)₂S | CH₃SH | CH₄ | (CH₃)₂S | CH₃SH | CH₄ |
| 300 | 17.7 | 100 | 0 | 38 | 100 | 0 | 71.1 | 99.9 | 0.1 |
| 325 | 28 | 100 | 0 | 53 | 99.9 | 0.1 | 72.5 | 99.6 | 0.4 |
| 350 | 35.1 | 100 | 0 | 72 | 99.8 | 0.2 | 72.1 | 98.1 | 1.9 |

It is noted that the catalysts according to the invention make it possible to significantly increase the conversion of the dimethyl sulfide, while at the same time maintaining very good selectivity. Notably, with the catalysts according to the invention, the conversion may be greater than 70%, relative to a maximum of 35% for alumina, which is the conventional catalyst used in sulfhydrolysis.

It is also noted that only a negligible amount of methane is formed.

### Example 2: Production of Mercaptans

The influence of the sulfhydrolysis reaction on the main mercaptan synthesis reaction was evaluated.

To do this, the content of sulfides coproduced by the main mercaptan synthesis reaction was monitored as a function of the variation in the content of mercaptans entering the reactor 1 (where the mercaptans are produced from alcohol and H₂S).

The conditions of the main reaction are as follows:

Pressure=3.5 bar

H₂S/ethanol ratio (molar)=2.4

GHSV=580 h⁻¹

The results for the selectivity toward sulfides obtained for three contents of mercaptans entering at three different temperatures with an alumina-based catalyst are described in FIG. 3.

As shown in FIG. 3, the selectivity toward sulfides coproduced during the main mercaptan synthesis reaction (Reactor 1) remains independent of the content of entering mercaptans, obtained from the sulfhydrolysis reaction. The absence of influence of the presence of mercaptans on the main synthetic reaction is thus demonstrated.

1. A sulfhydrolysis process, wherein a dialkyl sulfide is reacted with hydrogen sulfide (H₂S) in the presence of ZrO₂ and/or TiO₂ as catalyst(s), to obtain at least one mercaptan.

2. The process according to claim 1, wherein said catalyst(s) do not comprise any alkali metal oxide.

3. The process according to claim 1, said process being performed in a reactor comprising only one catalytic zone.

4. The process according to claim 1, wherein the reaction temperature is between 100° C. and 500° C.

5. The process according to claim 1, wherein the H₂S/dialkyl sulfide mole ratio is between 0.1/1 and 50/1.

6. The process according to claim 1, wherein the dialkyl sulfide is selected from the group consisting of: dimethyl sulfide, diethyl sulfide, dioctyl sulfide, didodecyl sulfide and methyl ethyl sulfide.

7. A process for preparing mercaptan(s), comprising:
preparing mercaptan(s) and dialkyl sulfide(s) from at least one alcohol and H₂S, and
reacting said dialkyl sulfide(s) produced with H₂S according to the sulfhydrolysis process as defined in claim 1, to obtain said mercaptan(s).

8. The process according to claim 7, comprising:
A) introducing a stream comprising H₂S and a stream comprising at least one alcohol into a first reactor;
B) reacting the two streams to obtain an outlet stream comprising at least one mercaptan, at least one dialkyl sulfide and optionally H₂S;
C) separating the outlet stream obtained from step B) into:
a stream comprising the mercaptan(s),
a stream comprising the dialkyl sulfide(s), and
optionally a stream comprising H₂S;
D) introducing the stream comprising the dialkyl sulfide(s) into a second reactor with a stream of H₂S;
E) reacting the two streams according to the sulfhydrolysis process to obtain an outlet stream comprising said mercaptan(s) and optionally H₂S;
F) optionally recycling the stream of H₂S obtained from step C) into step A).

9. The process according to claim 8, wherein the outlet stream from the second reactor of step E) is recycled into the first reactor of step A).

10. The process according to claim 7, comprising:
a) introducing a stream comprising a mercaptan and H₂S and a stream comprising an alcohol into a first reactor;
b) reacting the two streams to obtain an outlet stream comprising the mercaptan, a dialkyl sulphide and optionally H₂S;
c) separating the outlet stream obtained from step b) into:
a stream comprising the mercaptan;
a stream comprising the dialkyl sulphide; and
optionally a stream comprising H₂S;
d) introducing the stream comprising the dialkyl sulphide into a second reactor with a stream of H₂S;
e) reacting the two streams according to the sulfhydrolysis process to obtain an outlet stream comprising the mercaptan and H₂S;
f) introducing the outlet stream obtained from step e) into the reactor of step a);
g) optionally recovering the stream comprising the mercaptan obtained from step c); and
h) optionally recycling the stream of H₂S obtained from step c) into step a).

11. The use of ZrO₂ and/or TiO₂ as catalyst(s) in a reaction reacting a dialkyl sulfide with hydrogen sulfide to obtain a mercaptan.
